(19) United States
(12) Patent Application Publication
(10) Pub. No.: US 2018/0140527 A1
(43) Pub. Date: May 24, 2018

(54) ANTIMICROBIAL PEPTIDE STIMULATING CLEANSING COMPOSITION

(71) Applicant: GOJO Industries, Inc., Akron, OH (US)

(72) Inventors: Kegui Tian, Hudson, OH (US); Jessica Rae Tittl, Akron, OH (US)

(21) Appl. No.: 15/819,669

(22) Filed: Nov. 21, 2017

Related U.S. Application Data
(60) Provisional application No. 62/425,730, filed on Nov. 23, 2016.

(51) Int. Cl. A61K 8/64; A61Q 19/10; A61K 8/9789; A61Q 19/00; A61K 8/46

(52) U.S. Cl. CPC A61K 8/64; A61Q 19/10; A61K 8/463; A61Q 19/007; A61K 8/9789

(57) ABSTRACT

A method of increasing antimicrobial peptide production and/or activity on the skin is provided. The method includes cleaning skin with at least one of a soap and lotion and applying a topical composition to the skin. The topical composition is comprised of one or more polypeptides and extracts that increase the concentration of antimicrobial peptides on a surface.

### RELATED APPLICATIONS

This application claims priority to and the benefit of U.S. Provisional Patent Application Ser. No. 62/425,730, entitled “ANTIMICROBIAL PEPTIDE STIMULATING CLEANSING COMPOSITION” and filed Nov. 23, 2016, the entire disclosure of which is incorporated herein by reference.

### BACKGROUND

Skin disinfecting and cleansing compositions have become increasingly popular in the health care industry as well as with the general public for providing antimicrobial effectiveness to the skin without irritation.

Recent microbiome studies have analyzed the chemical make-up of the skin and the potential for disinfecting and cleansing compositions to improve both skin defense against germs and skin's innate immunity. This includes germ control through both internal and external methods. External methods include hygiene products that directly kill or slow germ growth. Internal methods include improving an organism's immune system to fight germs itself.

Antimicrobial peptides (“AMPs”), also known as host defense peptides, comprise a wide range of natural and synthetic peptides that are made of oligopeptides containing a varying number of amino acids. AMPs are essential components of host defense against infections present in all domains of life. AMPs are produced by all complex organisms and have diverse and intricate antimicrobial activities. As a whole, these peptides demonstrate a broad range of antiviral and antibacterial activities through an array of modes of action. AMPs have been found to kill Gram-negative and Gram-positive bacteria, certain viruses, parasites and fungi. Some research suggests that they can also enhance the internal immunity of complex organisms against a broad range of bacteria and viruses. In addition to the innate immune system present in all animals, vertebrates evolved an adaptive immune system based on specific recognition of antigens. Increasing evidence suggests that AMPs released in response to an invasion of microbial can activate adaptive immunity by attracting antigen-presenting dendritic cells to the invasion site.

While traditional soap and lotion formulations can stimulate the production of AMPs on the skin, the levels thereof are not sufficient to produce the desired effects of long lasting germ defense and innate immunity on the skin. It is thus desirable to design a new soap and/or lotion composition that is safe for topical use that stimulates the production AMPs to levels that help the skin fight germs and maintain continued immunity.

### SUMMARY

According to some exemplary embodiments, a composition for increasing the production and/or activity of antimicrobial peptides is provided. The topical cleansing composition includes about 0.005 wt. % to about 15.0 wt. % of an active ingredient that is one or more of an extract and a polypeptide. The topical cleansing composition also includes at least one primary and at least one secondary surfactant. The application of the topical cleansing composition increases the production and/or activity of antimicrobial peptides on the surface of the skin by an amount that is statistically significant compared to an otherwise identical composition without the active ingredient.

In some exemplary embodiments, the primary surfactant is sodium laureth sulfate and the secondary surfactant comprises cocamidopropyl betaine, disodium cocoamphodiacetate, cocamidopropyl hydroxysultaine, lauryl glucoside, and combinations thereof.

In some exemplary embodiments, the active ingredient is an extract that is one or more of a plant extract, a seed extract and a fruit extract. In other embodiments, the seed extract is at least one of linseed extract, flaxseed extract, hemp seed extract, grape seed extract, and grapefruit seed extract.

In some exemplary embodiments, the active ingredient is a hydrolysate of proteins, which can be proteins extracted from linseed seeds. The hydrolysate of linseed proteins can contain from about 0.1 to about 5.0 g/l of peptide compounds and from about 0.1 to 2.0 g/l of sugar. The peptide compounds can have a molecular weight below about 5.0 kDa.

In some exemplary embodiments, the active ingredient is a polypeptide that is one or more of an oligopeptide and a hexapeptide.

In some exemplary embodiments, the topical cleansing composition comprises from about 0.05 to about 5.0 wt. % or from about 0.1 to about 1.0 wt. % of the active ingredient, based on the weight of the topical cleansing composition.

In some exemplary embodiments, the topical cleansing composition further comprises one or more skin conditioning agents.

In some exemplary embodiments, the topical cleansing composition also contains up to about 20.0 wt. % of a humectant comprising propylene glycol, hexylene glycol, 1,4-dihydroxyhexane, 1,2,6-hexanetriol, sorbitol, butylene glycol, caprylyl glycol, propanediol s, such as methyl propane diol, dipropylene glycol, triethylene glycol, glycerin (glycerol), polyethylene glycols, ethoxydiglycol, polyethylene sorbitol, glyceryl caprylate/caprate, and combinations thereof.

In some exemplary embodiments, the topical cleansing composition also contains up to 10.0 wt. % of a moisturizing ester, comprising cetyl myristate, cetyl myristoleate, and other cetyl esters, diisopropyl sebacate, isopropyl myristate, and combinations thereof.

In some exemplary embodiments, the topical cleansing composition increases the production and/or activity of at least one anti-microbial peptide by a statistically significant amount. In some exemplary embodiments, the topical cleansing composition increases the production and/or activity of defenins by at least about 7%, or at least about 18%, or at least about 20%, or at least about 4 pg/mL, or at least about 25 pg/mL. In some exemplary embodiments, the topical cleansing composition increases the production and/or activity of chemokines by at least about 30%. In some exemplary embodiments, the topical cleansing composition increases the production and/or activity of cathelicidin-related antimicrobial peptides by at least about 32%. All percentages are relative to an otherwise identical topical composition without the active ingredient.

In some exemplary embodiments, the topical cleansing composition further comprises a carrier, which can be water.

In another exemplary embodiment, a skin treatment method for increasing the production and/or activity of antimicrobial peptides is provided. The method includes applying a topical cleansing composition to a skin surface, wherein the topical cleansing composition includes about 0.005 wt. % to about 15.0 wt. % of an active ingredient. The active ingredient may be one or more of an extract and a polypeptide. The topical cleansing composition also includes at least one primary surfactant and at least one secondary surfactant. The application of the topical cleansing composition increases the production and/or activity of AMPS on the surface of the skin by an amount that is statistically significant compared to an otherwise identical composition without the active ingredient.

### BRIEF DESCRIPTION OF THE FIGURES

FIG. 1 graphically illustrates HBD-1 concentrations after treatment with various concentrations of Decorinyl and Pamitoyl Pentapeptide-3.

FIG. 2 graphically illustrates HBD-2 concentrations after treatment with various concentrations of Decorinyl and Pamitoyl Pentapeptide-3.

FIG. 3 graphically illustrates HBD-3 concentrations after treatment with various concentrations of Decorinyl and Pamitoyl Pentapeptide-3.

FIG. 4 graphically illustrates HBD-1 concentrations after treatment with 0.1% and 1.0% Lipigenine™.

FIG. 5 graphically illustrates HBD-2 concentrations after treatment with 0.1% and 1.0% Lipigenine™.

FIG. 6 graphically illustrates HBD-3 concentrations after treatment with 0.1% and 1.0% Lipigenine™.

FIG. 7 graphically illustrates LL-37 concentrations after treatment with 0.1% and 1.0% Lipigenine™.

FIG. 8 graphically illustrates IL-8 concentrations after treatment with 0.1% and 1.0% Lipigenine™.

FIG. 9 graphically illustrates HBD-1 concentrations after treatment with various ingredients.

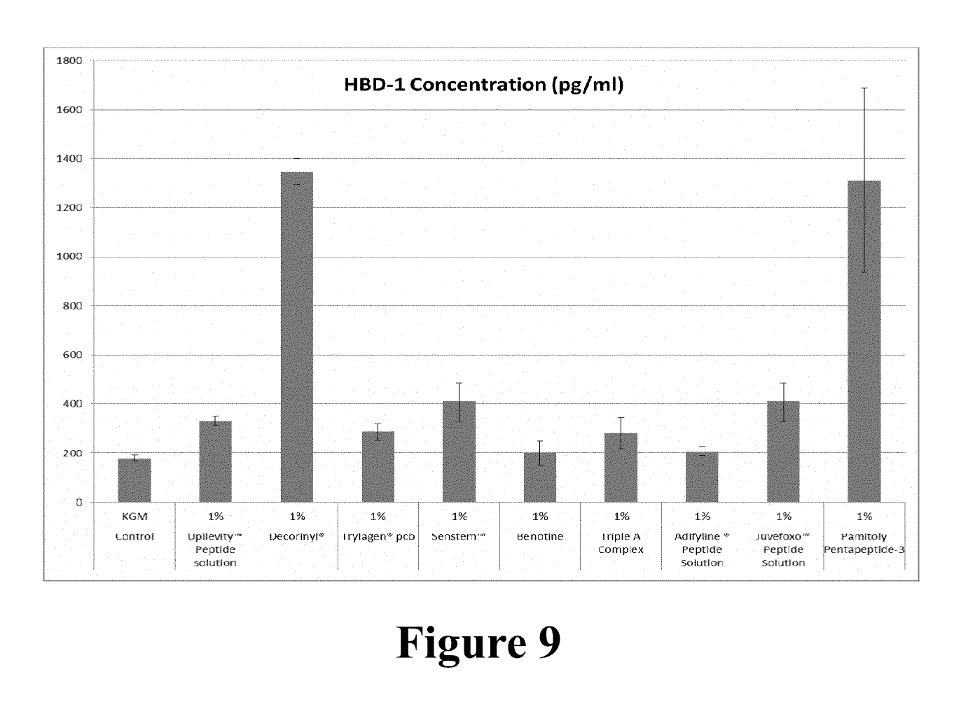

FIG. 10 graphically illustrates HBD-2 concentrations after treatment with various ingredients.

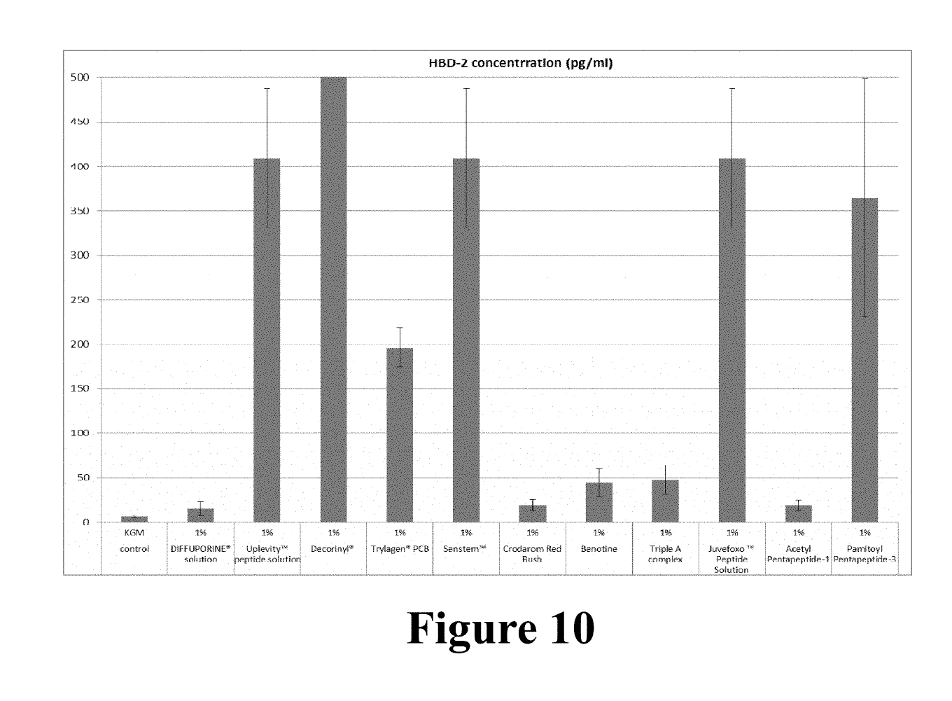

FIG. 11 graphically illustrates HBD-3 concentrations after treatment with various ingredients.

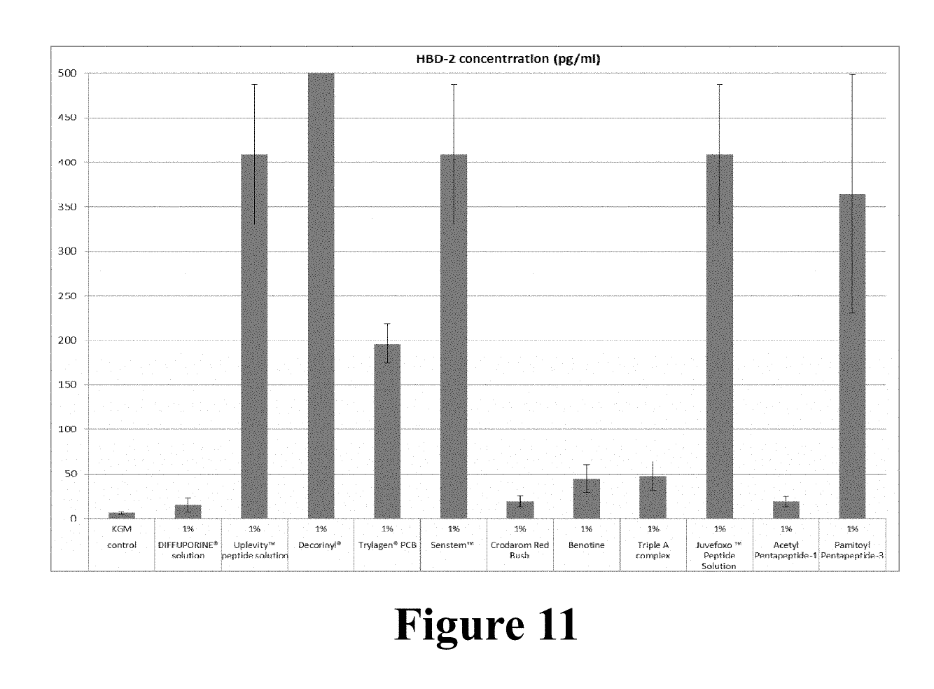

### DETAILED DESCRIPTION

Unless otherwise defined, all technical and scientific terms used herein have the same meaning as commonly understood by one of ordinary skill in the art to which this application pertains. Although other methods and materials similar or equivalent to those described herein may be used in the practice or testing of the exemplary embodiments, exemplary suitable methods and materials are described below. In case of conflict, the present specification including definitions will control. In addition, the materials, methods, and examples are illustrative only and not intended to be limiting of the general inventive concepts.

The terminology as set forth herein is for description of the exemplary embodiments only and should not be construed as limiting the application as a whole. Unless otherwise specified, “a,” “an,” “the,” and “at least one” are used interchangeably. Furthermore, as used in the description of the application and the appended claims, the singular forms “a,” “an,” and “the” are inclusive of their plural forms, unless contradicted by the context surrounding such.

The phrase “statistically significant” means p<0.05 for a test composition vs. a control that does not contain the active ingredient. The analysis is completed using 1) a T-test (a statistical examination of two population means) when only comparing one test article vs. one control); or 2) an analysis of variance (ANOVA) test when comparing two or more test articles vs. controls.

The phrase “topical composition” means a composition suitable for application directly to a surface, such as the surface of a human or animal body, including skin, and/or other surfaces, such as hair and nails.

The terms “polypeptide” and “polypeptides” as used herein refer to a chain of amino acids with two or more peptide bonds. In this way, these terms are meant to encompass both oligopeptides (which are generally considered to be peptide chains with between two and ten amino acids) as well as polypeptides (which are generally considered to be peptide chains with more than 10 amino acids).

The general inventive concepts relate to a topical composition that contains an AMP-stimulating active ingredient, including an extract and/or one or more polypeptides. In some exemplary embodiments, the active ingredient is an extract. The extract can be a modified extract, an unmodified extract, or an extract derivative. In one exemplary embodiment, the active ingredient is a natural extract, and can be derived from a plant extract, a fruit extract, and/or a seed extract. Non-limiting examples of natural extracts may include seed extracts, fruit extracts, linseed extract, flaxseed extract, hemp seed extract, grape seed extract, grapefruit seed extract, watermelon fruit extract, apple fruit extract, lentil fruit extract, hibiscus flower extract, pear fruit extract, root extract, leaf extract, Schinus terebinthifolius Seed Extract, Ascophyllum nodosum extract, soybean extract, Crothmum martimum extract, Lavandula stoechas extract, stem extracts, Sapindus Mukurossi fruit extract, sandalwood extract, bark extract, barley extract, Polygonum fagopyrum seed extract, avocado extract, cranberry fruit extract, blueberry fruit extract, Silena uniforla extract, Rosa multiflora extract, Evodia rutaecarpa fruit extract, algae extract, licorice leaf extract, jobi seed extract, seed oils, rosemary extract, green tea extract, plankton extract, himanthalia elongata extract, unidaria pinnatifida extract, Chlorella vulgaris extract, mugwort extract, and the like.

In some exemplary embodiments, the extract can be produced from the hydrolysis of natural proteins, which is referred to as a hydrolysate of proteins. Thus, the natural extracts may themselves comprise one or more peptides and/or polypeptides or the active ingredient may comprise peptides and/or polypeptide(s) independently. The hydrolysate can be obtained through hydrolysis of any type of protein, including proteins from any source. In some exemplary embodiments, the extract is a hydrolysate of linseed proteins, which are the proteins extracted from linseed seeds. Preferably, the linseed extract contains from about 0.1 to about 5.0 g/l of peptide compounds by weight of the dry extract and from about 0.1 to about 2.0 g/l of sugar by weight of the dry extract. These peptide compounds preferably have a molecular weight below about 5.0 kDa or below about 2.5 kDa.

The proteins can be any type of protein and can come from any type or part of a plant. In some exemplary embodiments, the plant can be of the Malpighiales order, of the Liaceae family, and/or of the Linum genus (linseed). Any method of extraction and purification can be employed to procure and prepare the protein extract.

In some exemplary embodiments, the natural extract is selected from one or more of the following compositions: (1) glycerin, plantago lanceolata leaf extract and xanthan gum (sold under the trade name Senestem™ by Sederma); (2) Benoitine (plankton extract in water); (3) water, glycerin, and hydrolyzed pearl (sold under the trade name Crodarom® by Croda Inc.) (4) Red Bush (rooibos) plant extract, (5) Phyko-Al-PF (water and hydrolyzed algin), and water, glycerin, and linseed (linum usitatissimum) seed extract (sold under the trade name Lipigenine™ by Ashland Chemical Company).

In some exemplary embodiments, the active ingredient comprises one or more peptides. Peptides are biologically-occurring short chains of amino acid monomers joined together by amide (peptide) bonds, which are formed through condensation reactions.

In other exemplary embodiments, the active ingredient comprises one or more oligopeptides. Oligopeptides are generally defined as peptide chains with 10 or fewer amino acids. In this way, the oligopeptide may be include, but is not limited to, an oligopeptide, such as a dipeptide, a tripeptide, a tetrapeptide, a pentapeptide, a hexapeptide, a heptapeptide, an octapeptide, a nonapeptide, and a decapeptide.

In other exemplary embodiments, the active ingredient comprises one or more polypeptides. A polypeptide is a long, continuous, unbranched peptide chain. Polypeptides are generally defined as peptide chains with more than 10 amino acids. The polypeptides of the exemplary embodiments described herein are not particularly limited and can be made of any number of peptide bonds.

In other exemplary embodiments, the active ingredient comprises a protein, which includes at least one long polypeptide that is arranged in a biologically functional way. The proteins of the exemplary embodiments described herein are not particularly limited and can include any number of polypeptides arranged in any biologically active manner. The peptides, oligopeptides, polypeptides, and proteins comprising the subject topical composition can be natural or synthetic peptides or polypeptides. They can further be modified or unmodified.

Exemplary polypeptides include Juvefoxo™; tetrapeptides, such as Uplevity™ Relistase®, and Decorinyl®; pentapeptides, such as palmitoyl pentapeptide-4, palmitoyl pentapeptide-3, and acetyl pentapeptide-1; hexapeptides, such as Adifyline® and acetyl hexapeptides; and mixtures of polypeptides and natural extracts, such as Triple A Complex, Trylagen® PCB. Exemplary acetyl hexapeptides include acetyl hexapeptide-1, acetyl hexapeptide-3, acetyl hexapeptide-7, acetyl hexapeptide-8, acetyl hexapeptide-19, acetyl hexapeptide-20, acetyl hexapeptide-22, acetyl hexapeptide-24, acetyl hexapeptide-30, acetyl hexapeptide-31, acetyl hexapeptide-37, acetyl hexapeptide-38, acetyl hexapeptide-39, acetyl hexapeptide-46, and acetyl hexapeptide-49. In some exemplary embodiments, the polypeptides include two or more acetyl hexapeptides.

In some exemplary embodiments, the topical cleansing composition disclosed herein includes an effective amount of active ingredient to increase the production and/or activity of at least one antimicrobial peptide on, for example, the skin. The topical cleansing composition can increase the production and/or activity of a wide variety of antimicrobial peptides, such as, for example defensins and cathelicidin-related AMPs and decrease pro-inflammatory factors. Such increased production and/or activity helps the skin's ability to defend against germs and helps improve the skin's innate immunity.

In one exemplary embodiment, the topical cleansing composition increases the production and/or activity of defensins. Defensins are cationic proteins that function as host defense peptides that have been found in vertebrates, invertebrates, and some plants. Defenins include at least α-defensins, β-defensins, and β-defensins. In some exemplary embodiments, the topical composition increases the production and/or activity of β-defensins, such as HBD-1, HBD-2, and HBD-3.

In some exemplary embodiments, the topical cleansing composition increases the production and/or activity of cathelicidin-related antimicrobial peptides. Cathelicidins play a vital role in mammalian innate immunity against invasive bacterial infections. In some exemplary embodiments, the topical cleansing composition increases the production and/or activity of the cathelcidin-related AMP, LL-37.

In other exemplary embodiments, the topical cleansing composition decreases the production and/or activity of pro-inflammatory factors. In some exemplary embodiments, the topical cleansing composition increases the production and/or activity of the pro-inflammatory factor, chemokines, such as IL-8.

Traditionally, it has been found that compositions used to stimulate the production and/or activity of AMPs also cause skin inflammation and/or skin irritation. However, it has been discovered that a topical cleansing composition comprising the subject active ingredient is capable of increasing the production and/or activity of at least one AMP on the skin without causing irritation/inflammation of the skin.

The effective amount of active ingredient in the topical cleansing composition may include up to about 15.0 percent by weight (wt. %) of the active ingredient, based on the weight of the topical cleansing composition. In some exemplary embodiments, the effective amount of active ingredient comprises about 0.02 to about 5.0 wt. %, or from about 0.5 to about 2.0 wt. %, based on the weight of the topical cleansing composition. In other exemplary embodiments, the effective amount of active ingredient comprises about 0.1 to about 1.0 wt. %, based on the weight of the topical cleansing composition.

In some exemplary embodiments, the topical cleansing composition is in the form of a cleanser, such as a soap or a lotion-based cleanser and is used for application to the skin. The topical cleansing composition may be in the form of a skin cleanser, skin moisturizer, skin protectant, shampoo, a wipe, a lotion, a salve, foam, soap, gel, a cream, etc. A wide variety of vehicles may be used to deliver the topical composition, such as, for example pads, bandages, patches, sticks, aerosol dispersers, pump sprays, trigger sprays, canisters, foam pumps, wipes, and the like. The topical cleansing composition may be applied to the skin before, during, or after skin cleaning.

In some exemplary embodiments, the topical cleansing composition comprises a carrier. The carrier can be any suitable compound able to effectively deliver and/or transport the topical composition. In some exemplary embodiments, the carrier is water or a base cleaner. In other exemplary embodiments, the topical cleansing composition does not include any carrier and is delivered as a concentrate.

In some exemplary embodiments, the topical cleansing composition includes water as the carrier in an amount quantum sufficit (q.s.). In some exemplary embodiments, the topical cleansing composition comprises at least about 40.0 weight percent (wt. %) water, in another embodiment, the topical composition comprises at least about 50.0 wt. % water, in another embodiment, the topical composition comprises at least about 60.0 wt. % water, in another embodiment, the topical composition comprises at least about 70.0 wt. % water, in another embodiment, the topical composition comprises at least about 80.0 wt. % water, and in yet another embodiment, the topical composition comprises at least about 83.0 wt. % water, and in still yet another embodiment, the topical composition comprises at least about 85.0 wt. % water, based on the weight of the topical cleansing composition. In other embodiments, the topical composition comprises from about 80.0 wt. % to about 90.0 wt. % water, based on the weight of the topical cleansing composition. In a preferred embodiment, the topical composition comprises from about 83.0 to about 87.0 wt. % water, based on the weight of the topical cleansing composition. More or less water may be required in certain instances, depending particularly on other ingredients and/or the amounts thereof employed.

In one or more embodiments, the topical cleansing composition includes one or more skin-conditioners. Various classes or types of skin-conditioners have been used such as humectants, emollients, and other miscellaneous compounds which exhibit occlusive properties upon application to the skin. Non-limiting examples of suitable skin conditioners and emollients include aloe, vitamin E, vitamin E acetate (tocopheryl acetate), Vitamin B₃ (niacinamide), C₆₋₁₀ alkane diols, sodium salt of pyroglutamic acid (sodium PCA), PEG-7 glyceryl cocoate, coco-glucoside and/or glyceryl oleate (Lamisoft® PO), and polyquaternium, such as polyquaternium 10 and 39.

If an emollient or one of the miscellaneous skin-conditioners, such compound can be included in the topical cleansing composition in an amount from about 0.0001 to about 10.0 wt. %, in other embodiments, from about 0.0005 to about 5.0 wt. %, based on the weight of the topical cleansing composition. In one exemplary embodiment, the miscellaneous skin conditioner is present in an amount from about 0.1 to about 2.0 wt. %, based on the weight of the topical cleansing composition and in yet another exemplary embodiment, from about 0.5 to about 1.0 wt. %, based on the weight of the topical cleansing composition.

In some exemplary embodiments, the topical cleansing composition includes one or more humectants as the skin conditioner. Non-limiting examples of humectants include propylene glycol, hexylene glycol, 1,4-dihydroxyhexane, 1,2,6-hexanetriol, sorbitol, butylene glycol, caprylyl glycol, propanediols, such as methyl propane diol, dipropylene glycol, triethylene glycol, glycerin (glycerol), polyethylene glycols, ethoxydiglycol, polyethylene sorbitol, glycerol caprylate/caprate (GCC), and combinations thereof. Other humectants include glycolic acid, glycolate salts, lactate salts, urea, Jojoba wax PEG-120 esters (commercially available from FloraTech), hydroxyethyl urea, alpha-hydroxy acids, such as lactic acid, sodium pyrrolidone carboxylic acid, hyaluronic acid, chitin, and the like. In one exemplary embodiment, the humecant is a mixture of caprylyl glycol, sodium L-pyroglutamate (Sodium PCA), and glycerin.

Examples of polyethylene glycol humectants include PEG-4, PEG-6, PEG-7, PEG-8, PEG-9, PEG-10, PEG-12, PEG-14, PEG-16, PEG-18, PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, and PEG-800.

The humectant may be included in the topical cleansing composition in an amount up to about 20.0 wt. %, or up to about 15.0 wt. %, or up to about 12.0 wt. %, or up to about 10.0 wt. %, or up to about 8.0 wt. %, or up to about 3.0 wt. %, based on the weight of the topical cleansing composition. In some exemplary embodiments, the humectant is included in an amount from about 0.001 wt. %, or from about 0.01 wt. %, or from about 0.05 wt. %, or from about 0.1 wt. %, or from about 0.5 wt. %, or from about 0.7 wt. %, or from about 1.0 wt. %, or from about 1.5 wt. %, or from about 2.0 wt. %, based on the weight of the topical cleansing composition. In one exemplary embodiment, the humectant is included in an amount from about 0.4 to about 3.0 wt. %, or from about 1.5 to about 2.0 wt. %, based on the weight of the topical cleansing composition.

The topical cleansing composition may further comprise a plug-preventing additive. In some exemplary embodiments, the plug-preventing additive can also, as discussed above, act as the humectant. In one or more embodiments the plug-preventing comprises one or more diols, that is compounds with two hydroxyl groups. Plug-preventing additives that contain more or less hydroxyl groups (i.e., one hydroxyl group or three or more hydroxyl groups) are also within the purview of the exemplary embodiments described herein. In one or more exemplary embodiments the diol is a C₆₋₁₀ alkane diol and in some exemplary embodiments, a straight chain C₆₋₁₀ alkane diol, that is, a straight chain diol with a chain of 6 to 10 carbon atoms. Non-limiting examples of suitable diols include 1,2-hexanediol, 1,2-octanediol (often referred to as caprylyl glycol), 1,9-nonanediol, 1,2-decanediol, 1,10-decanediol, or mixtures and blends thereof. The diol can contain any other functional groups including, for example, esters, carboxylic acids, ethers, amides, amines, alkyl halides, phenyls, as well as other carbonyl-containing functional groups. In some exemplary embodiments, the plug-preventing agent contains at least one ester and/or at least one amide group. Non-limiting examples of such compounds include glycerol caprylate/caprate and cocoamide diethanolamine.

If separate from the humectant, the plug-preventing additive may be included in the topical cleansing composition in an amount up to about 20.0 wt. %, or up to about 15.0 wt. %, or up to about 12.0 wt. %, or up to about 10.0 wt. %, or up to about 8.0 wt. % or up to about 5.0 wt. %, or up to about 3.0 wt. %, based on the weight of the topical cleansing composition. In some exemplary embodiments, the plug-preventing agent is included in an amount from about 0.001 wt. %, or from about 0.01 wt. %, or from about 0.05 wt. %, or from about 0.1 wt. %, or from about 0.5 wt. %, or from about 0.7 wt. %, or from about 1.0 wt. %, or from about 1.5 wt. %, or from about 2.0 wt. %, based on the weight of the topical cleansing composition. In one exemplary embodiment, the plug-preventing additive is included in an amount from about 0.05 to about 4.0 wt. %, or from about 0.1 to about 1.0 wt. %, or from about 0.15 to about 0.7 wt. %, or from about 0.2 to about 0.7 wt. %, based on the weight of the topical cleansing composition.

In certain embodiments, the diol plug-preventing additive is added to the topical cleansing composition as a solution or emulsion. That is, the diol can be premixed with a carrier to from a diol solution or emulsion, with the proviso that the carrier does not deliriously effect the ability of the topical cleansing composition to sanitize and increase the production or activity of antimicrobial peptides. Non-limiting examples of carriers include, water, alcohol, glycols such as propylene or ethylene glycol, ketones, linear and/or cyclic hydrocarbons, triglycerides, carbonates, silicones, alkenes, esters such as acetates, benzoates, fatty ester, glyceryl esters, ethers, amides, polyethylene glycol, and PEG/PPG copolymers, inorganic salts solutions such as saline, and mixtures and blends thereof.

In some exemplary embodiments, the topical cleansing composition further comprises one or more conditioning or moisturizing esters. Examples of such conditioning or moisturizing esters include cetyl myristate, cetyl myristoleate, and other cetyl esters, diisopropyl sebacate, and isopropyl myristate. The ester may be present in an amount of up to about 10.0 wt. %, or up to about 8.0 wt. %, or up to about 5.0 wt. %, or up to about 3.0 wt. %, or up to about 2.0 wt. %, or up to about 1.0 wt. %, based on the weight of the topical cleansing composition. In some exemplary embodiments, the moisturizing ester is present in an amount from about 0.001 wt. %, or from about 0.005 wt. %, or from about 0.01 wt. %, or from about 0.05 wt. %, or from about 0.1 wt. %, or from about 0.5 wt. %, or from about 1.0 wt. %, based on the weight of the topical cleansing composition. In one exemplary embodiment, the moisturizing ester is present in an amount between 0.01 to 0.30 wt. %, based on the weight of the topical cleansing composition. In another exemplary embodiment, the moisturizing ester is present in an amount between 0.05 wt. % and 0.25 wt. %, based on the weight of the topical cleansing composition.

In some exemplary embodiments, the topical cleansing composition further comprises one or more deposition enhancers. A suitable deposition enhancer works unidirectionally and will allow ingredients within the composition to penetrate deeper into the stratum corneum whilst preventing the loss of materials from the skin. Advantageously, the deposition enhancer provides a cosmetically acceptable skin feel to the formulation.

In one or more embodiments, the deposition enhancers include one or more of surfactants, bile salts and derivatives thereof, chelating agents, and sulphoxides.

Some examples of acceptable deposition enhancers include hydroxypropyl methylcellulose, dimethyl sulphoxides (DMSO), DMA, DMF, 1-dodecylazacycloheptan-2-one (azone), pyrrolidones such as 2-Pyrrolidone (2P) and N-Methyl-2-Pyrrolidone (NMP), long-chain fatty acids such as oleic acid and fatty acids with a saturated alkyl chain length of about C₁₀-C₁₂, essential oils, terpenes, terpenoids, oxazolidinones such as 4-decyloxazolidin-2-one, sodium lauryl sulfate (SLS), sodium laureate, polysorbates, sodium glyacolate, sodium deoxycholate, caprylic acid, EDTA, phospholipids, C₁₂₋₁₅ Alkyl Benzoate, pentylene glycol, ethoxydiglycol, polysorbate-polyethylenesorbitan-monolaurate, and lecithin.

In one or more exemplary embodiments, the deposition enhancer is a quaternary ammonium compound such as polyquaternium-6, -7, -10, -22, -37, -39, -74 or -101.

In some exemplary embodiments the deposition enhancer is included in the topical cleansing composition in an amount from about 0.005 wt. % to about 10.0 wt. %, in other embodiments, from about 0.01 wt. % to about 5.0 wt. %, and in other embodiments, from about 0.05 wt. % to about 3.0 wt. %, based on the weight of the topical cleansing composition.

In one or more exemplary embodiments, the deposition enhancer comprises a hydroxy-terminated polyurethane compound chosen from polyolprepolymer-2, polyolprepolymer-14, and polyolprepolymer-15. Polyolprepolymer-2 is sometimes referred to as PPG-12/SMDI copolymer. The polyurethane compound may be present in the topical cleansing composition in an amount from about 0.005 wt. % to about 5.0 wt. %, in other embodiments, from about 0.01 wt. % to about 3.0 wt. %, and in other embodiments, from about 0.05 wt. % to about 1.0 wt. %, based on the weight of the topical cleansing composition.

In some exemplary embodiments, the topical composition further comprises one or more preservatives. A preservative is a natural or synthetic ingredient that can be added to personal care products to prevent spoilage, such as from microbial growth or undesirable chemical changes. Typical cosmetic preservatives are classified as natural antimicrobials, broad-spectrum preservatives, or stabilizers.

Many different types of preservatives are envisioned as being applicable in the current topical composition. Non-limiting examples of preservatives include one or more of isothiazolinones, such as methylchloroisothiazolinone and methylisothiazolinone; parabens including butylparaben, propylparaben, methylparaben and germaben II; phenoxyetyhanol and ethylhexylglycerin, organic acids such as potassium sorbate, sodium benzoate and levulinic acid; and phenoxyethanols.

The preservative can be added in the topical cleansing composition in an amount up to about 10.0 wt. %, preferably from about 0.05 wt. % to about 5.0 wt. %, more preferably from about 0.1 wt. % to about 2.0 wt. %, based on the weight of the topical cleansing composition. In one exemplary embodiment, the preservative is present in an amount from about 1.0 to about 1.5 wt. %, based on the weight of the topical cleansing composition.

In some exemplary embodiments, the topical composition further comprises one or more anti-irritants. Anti-irritants reduce signs of inflammation on the skin such as swelling, tenderness, pain, itching, or redness. There are three main types of anti-irritants, all of which are envisioned as being applicable in the exemplary embodiments described herein: (1) compounds that operate by complexing the irritant itself, (2) compounds that react with the skin to block reactive sites preventing the irritant from reacting directly with the skin, and (3) compounds that prevent physical contact between the skin and irritant.

Some exemplary examples of suitable anti-irritants include Aloe Vera, allantoin, anion-cation complexes, aryloxypropionates, azulene, carboxymethyl cellulose, cetyl alcohol, diethyl phthalate, Emcol E607, ethanolamine, glycogen, lanolin, N-(2-Hydroxylthyl) Palmitamide, N-Lauroyl Sarcosinates, Maypon 4C, mineral oils, miranols, Myristyl lactate, polypropylene glycol, polyvinyl pyrrolidone (PVP), tertiary amine oxides, thiodioglycolic acid, and zirconia. In one exemplary embodiment, the anti-irritant is avenanthrmides (avena sativa (oat), kernel oil, and glycerin) and niacinamide.

In some exemplary embodiments, the anti-irritant is included in the topical cleansing composition in an amount up to about 10.0 wt. %, in other embodiments, from about 0.005 wt. % to about 3.0 wt. %, and in other embodiments, from about 0.01 wt. % to about 1.0 wt. %, based on the weight of the topical cleansing composition.

The topical cleansing composition may further comprise a fragrance. Any scent may be used in the topical composition including, but not limited to, any scent classification on a standard fragrance chart, such as floral, oriental, woody, and fresh. Exemplary scents include cinnamon, clove, lavender, peppermint, rosemary, thyme, thieves, lemon, citrus, coconut, apricot, plum, watermelon, ginger, and combinations thereof.

The fragrance can be included in the topical cleansing composition in an amount from about 0.005 wt. % to about 5.0 wt. %, in other embodiments, from about 0.01 wt. % to about 3.0 wt. %, and in other embodiments, from about 0.05 wt. % to about 1.0 wt. %, based on the weight of the topical cleansing composition. The fragrance can be any made of any perfume, essential oil, aroma compounds, fixatives, terpenes, solvents, and the like. In some exemplary embodiments, the essential oils may include, for example, one or more of Limonene, Citrus Aurantium Dulcis (Orange) Peel Oil, Eucalyptus Globulus Leaf Oil, Citrus Grandis (Grapefruit) Peel Oil, Linalool, Litsea Cubeba Fruit Oil, Lavandula Hybrida Oil, Abies Sibirica Oil, Mentha Citrata Leaf Extract, Coriandrum Sativum (Coriander) Fruit Oil, Piper Nigrum (Pepper) Fruit Oil, and Canarium Luzonicum Gum Nonvolatiles.

The topical cleansing composition may further comprise a wide range of optional ingredients that do not deleteriously affect the composition's ability to increase the production and/or activity of AMPS on the surface or the composition's ability to regulate the balance of bacteria on the skin. The CTFA International Cosmetic Ingredient Dictionary and Handbook, Eleventh Edition 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the exemplary embodiments described herein. Examples of these functional classes include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include solubilizing agents, sequestrants, keratolytics, topical active ingredients, and the like.

In some exemplary embodiments, the topical composition exhibits a pH in the range of from about 3.0 to about 12.0, or a pH in the range of from about 4 to about 8, or in the range of from about 4.5 and about 7.0. When necessary, a pH adjusting agent or constituent may be used to provide and/or maintain the pH of a composition. Exemplary pH adjusting agents include, but are not limited to, organic acids, such as citric acid, lactic acid, formic acid, acetic acid, proponic acid, butyric acid, caproic acid, oxalic acid, maleic acid, benzoic acid, carbonic acid, and the like.

The form of the topical cleansing composition according to the exemplary embodiments described herein is not particularly limited. In one or more embodiments, topical cleansing compositions according to the exemplary embodiments described herein may be formulated as a cleansing lotion, a foamable composition, a rinse-off soap cleansing composition, a thickened gel composition, or may be applied to a wipe.

In one or more embodiments, the topical cleansing composition is formulated as a foamable composition. One or more foam agents may optionally be included in the foamable composition.

Any foaming agent conventionally known and used may be employed in the topical cleansing composition. In one or more embodiments, the foam agent comprises a non-ionic foam agent such as decyl glucoside or an amphoteric foam agent such as cocamidopropylbetaine. In one or more embodiments, the amount of nonionic or amphoteric foam agent is from about 0.5 to about 3.5 wt. %, in other embodiments from about 1.0 to about 3.0 wt. %, based on the weight of the topical cleansing composition. In one or more embodiments, the amount of decyl glucoside or cocamidopropylbetaine is from about 0.5 to about 3.5 wt. %, in other embodiments from about 1.0 to about 3.0 wt. %, based on the weight of the topical cleansing composition.

In some exemplary embodiments, the foaming agents include one or more of silicone glycol and fluorosurfactants. Silicone glycols may be generally characterized by containing one or more Si—O—Si linkages in the polymer backbone. Silicone glycols include organopolysiloxane dimethicone polyols, silicone carbinol fluids, silicone polyethers, alkylmethyl siloxanes, amodimethicones, trisiloxane ethoxylates, dimethiconols, quaternized silicone glycols, polysilicones, silicone crosspolymers, and silicone waxes.

Examples of silicone glycols include dimethicone PEG-7 undecylenate, PEG-10 dimethicone, PEG-8 dimethicone, PEG-12 dimethicone, perfluorononylethyl carboxydecal PEG 10, PEG-20/PPG-23 dimethicone, PEG-11 methyl ether dimethicone, bis-PEG/PPG-20/20 dimethicone, silicone quats, PEG-9 dimethicone, PPG-12 dimethicone, fluoro PEG-8 dimethicone, PEG-23/PPG-6 dimethicone, PEG-20/PPG-23 dimethicone, PEG 17 dimethicone, PEG-5/PPG-3 methicone, bis-PEG-18 methyl ether dimethyl silane, bis-PEG-20 dimethicone, PEG/PPG-20/15 dimethicone copolyol and sulfosuccinate blends, PEG-8 dimethicone\dimmer acid blends, PEG-8 dimethicone\fatty acid blends, PEG-8 dimethicone\cold pressed vegetable oil\polyquaternium blends, random block polymers and mixtures thereof.

The amount of silicone glycol foam agent is not particularly limited, so long as an effective amount to produce foaming is present. In certain embodiments, the effective amount to produce foaming may vary, depending on the amount of other ingredients that are present. In one or more embodiments, the composition includes at least about 0.002 wt. % of silicone glycol foam agent, based on the weight of the topical cleansing composition. In another embodiment, the composition includes at least about 0.01 wt. % of silicone glycol foam agent, based on the weight of the topical cleansing composition. In yet another embodiment, the composition includes at least about 0.05 wt. % of silicone glycol foam agent, based on the weight of the topical cleansing composition.

In some exemplary embodiments, the foam agent is present in an amount of from about 0.002 to about 4.0 wt. %, or in an amount of from about 0.01 to about 2.0 wt. %, based on the weight of the topical cleansing composition. It is envisioned that higher amounts may also be effective to produce foam. All such weights as they pertain to listed ingredients are based on the active level, and therefore, do not include carriers or by-products that may be included in commercially available materials, unless otherwise specified.

In other embodiments, it may be desirable to use higher amounts of foam agent. For example, in certain embodiments where the foaming composition of the exemplary embodiments described herein includes a cleansing product that is applied to a surface and then rinsed off, higher amounts of foam agent may be employed. In these embodiments, the amount of foam agent is present in amounts up to about 35.0 wt. %, based on the weight of the topical cleansing composition.

In some exemplary embodiments, the topical cleansing composition is formulated as an aerosol or non-aerosol foamable composition. In some exemplary embodiments the topical cleansing composition is dispensed from an unpressurized or low-pressure dispenser which mixes the composition with air.

In one or more embodiments, the viscosity of the non-aerosol foamable composition is less than about 100 mPas, in one embodiment less than about 50 mPas, and in another embodiment less than about 25 mPas.

In one or more embodiments, the topical cleansing compositions is formulated as a lotion. As is known in the art, lotions include oil-in-water emulsions as well as water-in-oil emulsions, oil-water-oil, and water-oil-water. A wide variety of ingredients may be present in either the oil or water phase of the emulsion. That is, the lotion formulation is not particularly limited.

Compositions of the exemplary embodiments described herein may be characterized by reference to viscosity and/or rheological properties. In one or more embodiments, the viscosity may be expressed as a standard, single-point type viscosity, as measured on a Brookfield Digital viscometer at a temperature of about 20° C., using spindle T-D, heliopath, at a speed of 10 rpm. In one or more embodiments, the compositions may have a viscosity of from about 2,000 to about 120,000 centipoise (cP).

In one or more embodiments, compositions of the exemplary embodiments described herein may be characterized as lotions, having a viscosity of less than about 120,000 cP, in other embodiments, less than about 100,000, and in other embodiments, less than about 75,000 cP. In one or more embodiments, the lotion compositions may have a viscosity of from about 3,000 to about 50,000 cP, in other embodiments, from about 4,000 to about 30,000 cP.

Exemplary lotion formulations include those containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties.

In one or more embodiments, the topical cleansing composition is characterized as serum, having a viscosity of from about 2,000 to about 3000 cP.

In one or more embodiments, the topical cleansing composition is characterized as creams, having a viscosity of from about 30,000 to about 100,000 cP, in other embodiments from about 50,000 to about 80,000 cP.

In one or more embodiments, the topical cleansing composition is pourable at room temperature, i.e. a temperature in the range of from about 20 to about 25° C. In one or more embodiments, the lotion formulations are viscous enough to hold a shape or not flow for a desired period of time. In other embodiments, the topical cleansing composition is a cream or ointment, and are not pourable and do not flow at room temperature and will not conform to a container when placed into the container at room temperature.

In one or more embodiments, the topical cleansing composition includes thickeners and optionally one or more stabilizers. Examples of thickeners and stabilizers include polyurethane-based thickeners, such as steareth-100/PEG-136/HDI copolymer (Rheoluxe® 811); sodium chloride; propylene glycol; PEG-120 methyl glucose dioleate and methyl gluceth-10 (Ritathix DOE, available from Rita Corp.); hydroxyethyl cellulose; quaternized hydroxyethyl cellulose (Polyquaternium-10); hydroxypropyl cellulose; methyl cellulose; carboxymethyl cellulose; and ammonium acryloyldimethyltaurate/VP copolymer.

In one or more exemplary embodiments, the topical cleansing composition may be thickened with polyacrylate thickeners such as those conventionally available and/or known in the art. Examples of polyacrylate thickeners include carbomers, acrylates/C 10-30 alkyl acrylate cross-polymers, copolymers of acrylic acid and alkyl (C₅-C₁₀) acrylate, copolymers of acrylic acid and maleic anhydride, and mixtures thereof. In one or more embodiments, the gel composition includes an effective amount of a polymeric thickener to adjust the viscosity of the gel to a viscosity range of from about 1,000 to about 65,000 cP. In one embodiment, the viscosity of the gel is from about 5,000 to about 35,000 cP, and in another embodiment, the viscosity is from about 10,000 to about 25,000 cP. The viscosity is measured by a Brookfield RV Viscometer using RV and/or LV Spindles at 22° C.+/−3° C.

As will be appreciated by one of skill in the art, the effective amount of thickener will vary depending upon a number of factors, including the amount of other ingredients in the topical cleansing composition. In one or more embodiments, an effective amount of thickener is at least about 0.01 wt. %, based on the weight of the topical cleansing composition. In other embodiments, the effective amount is at least about 0.02 wt. %, or at least about 0.05 wt. %, or at least about 0.1 wt. %, based on the weight of the topical cleansing composition. In some exemplary embodiment, the effective amount of thickener is at least about 0.5 wt. %, or at least about 0.75 wt. %, based on the weight of the topical cleansing composition. In one or more embodiments, the topical cleansing composition comprises up to about 10.0 wt. % of a polymeric thickener, based on the weight of the topical cleansing composition. In certain embodiments, the amount of thickener is from about 0.01 to about 1.0 wt. %, or from about 0.02 to about 0.4 wt. %, or from about 0.05 to about 0.3 wt. %, based on the weight of the topical cleansing composition. The amount of thickener may be from about 0.1 to about 10.0 wt. %, or from about 0.5 to about 5.0 wt. %, or from about 0.75 to about 2.0 wt. %, based on the weight of the topical cleansing composition.

In one or more embodiments, the topical cleansing composition may further comprise a neutralizing agent. Examples of neutralizing agents include amines, alkanolamines, alkanolamides, inorganic bases, amino acids, including salts, esters and acyl derivatives thereof. Exemplary neutralizing agents include triethanolamine, sodium hydroxide, monoethanolamine and dimethyl stearylamine. Other neutralizing agents are also known, such as HO(CₘH₂ₘ)₂NH, where m has the value of from 2 to 3, and aminomethyl propanol, aminomethyl propanediol, and ethoxylated amines, such as PEG-25 cocamine, polyoxyethylene (5) cocamine (PEG-5 cocamine), polyoxyethylene (25) cocamine (PEG-25 cocamine), polyoxyethylene (5) octadecylamine (PEG-5 stearamine), polyoxyethylene (25) octadecylamine (PEG-25 stearamine), polyoxyethylene (5) tallowamine (PEG-5 tallowamine), polyoxyethylene (15) oleylamine (PEG-15 oleylamine), polyethylene (5) soyamine (PEG-5 soyamine), and polyoxyethylene (25) soyamine (PEG-15 soyamine). A number of these are commercially available under the trade name of Ethomeen® from Akzo Chemie America, Armak Chemicals of Chicago, Ill.

In some exemplary embodiments the neutralizing agent includes at least one of sodium hydroxide or sodium hydroxide precursors. Solutions of sodium hydroxide in water are non-limiting examples of neutralizers containing sodium hydroxide.

The neutralizing agent is employed in an effective amount to neutralize a portion of the carboxyl groups of the thickening agent, and produce the desired pH range. The pH of un-neutralized thickening agent dispersed in water is generally acidic. For example, the pH of Carbopol® polymer dispersions is approximately in the range of 2.5 to 3.5, depending upon the polymer concentration. An effective amount of neutralizing agent, when added to the thickener dispersion, adjusts the pH to a desired range of about 4.1 to 4.8, or of about 4.2 to 4.6. The amount of neutralizing agent necessary to effect this pH range will vary depending upon factors such as the type of thickening agent, the amount of thickening agent, etc. However, in general, amounts less than 1.0 wt. % or ranging from about 0.001 to about 0.3 wt. %, by weight of the neutralizing agent, are considered sufficient and effective.

In some exemplary embodiments the topical cleansing composition can also be formulated as a cleansing composition or soap. A fatty acid or a fatty acid ester may be used in conjunction with an alkali or base from the water phase to form a soap which has good water solubility as well as oil solubility properties and hence, is an excellent emulsifier. The soap, as explained above, can be in the form of a lotion soap, a foam soap, or any other common form known to one of skill in the art. Typical commercial blends such as oleic fatty acid, coconut fatty acid, soya fatty acid and tall oil fatty acid can be used. Preferably, the fatty acid comprises from about 5.0 to about 10.0 wt. %, based on the weight of the topical cleansing composition.

As explained above, a base may be utilized in conjunction with the fatty acid to produce a soap on an equivalent basis of from about 2.7 to 0.8 equivalents to 1 equivalent of base. Examples of suitable base include organic alkalis or amines such as monoethanolamine, triethanolamine, and mixed isopropanolamines such as diisopropanolamine. Examples of suitable base also include inorganic alkalis, such as potassium hydroxide, sodium hydroxide, ammonium hydroxide, soda ash, and ammonia.

In addition, one or more surfactants can be included in the oil phase of the topical cleansing composition in amounts preferably ranging up to about 25.0 wt. %, based on the weight of the topical cleansing composition. A surfactant is generally any substance which reduces the surface tension of a liquid. They break down the interface between water and oils/dirt. By holding the oils/dirt in suspension, they can be easily removed from the surface (i.e. skin).

In some exemplary embodiments, the surfactant includes a mixture of primary and secondary surfactants. Nonionic surfactants, i.e., surfactants which are uncharged (neutral) and without cationic or anionic sites, are preferred since they tend to render the composition stable, i.e., impart two desirable properties thereto. The first property is that of a suitable long shelf life. In other words, the emulsion can be held together at room temperature for long periods of time. The second desirable property is that upon use of the cleaning composition, the surfactant permits breakage of the emulsion or opening up thereof such that the hydrocarbon oil is readily released. The surfactant can also be an anionic surfactant, which carry a negative charge and are ionized in solution. The surfactant can also be a cationic surfactant, which carry a positive charge and ionize in solution. The surfactant can also be an amphoteric surfactant, which have the ability to be anionic (negatively charged), cationic (positively charged), or nonionic (uncharged, neutral) in solution depending on the pH.

It will be appreciated by one skilled in the art that in one or more embodiments, surfactant and/or surfactant combinations may be chosen to limit irritation of the topical cleansing composition and/or to enhance the effect of the active ingredient. In yet another embodiment, surfactant and/or surfactant combinations may be chosen to allow maximum bioavailability of the active ingredient. Non-limiting exemplary examples of surfactant combinations, levels of which will be known to one skilled in the art, are sodium lauryl ether sulfate (SLES) and/or cocamidopropyl betaine and/or disodium cocoamphodiacetate and/or surfactants of similar structure.

Non-limiting exemplary examples of surfactants that are envisioned in the present topical cleansing composition include betaines such as cocamidopropyl betaine; sulfonates and sulfates such as sodium laureth sulfate, sodium cocosulfate, sodium trideceth sulfate, and alkylbenzene sulfonate; glucosides, such as lauryl gluocoside and decyl glucoside; sodium cocoyl isothionate, sodium cocoyl glycinate, cocamidopropyl hydroxysultaine, PEG-80 sorbitan laurate, di-alkyl sulfosuccinate, lignosulfonates, disodium cocoamphodiacetate, lauryl glucoside, and PEG-80 sodium laurate.

In some exemplary embodiments, the topical cleansing composition comprises at least one primary surfactant and at least one secondary surfactant. A primary surfactant may include, for example, sodium laureth sulfate. Exemplary secondary surfactants may include, for example, one or more of cocamidopropyl betaine, disodium cocoamphodiacetate, cocamidopropyl hydroxysultaine, and lauryl glucoside.

As will be appreciated by one of skill in the art, the amount of surfactant will vary depending upon a number of factors, including the amount of other ingredients in the topical composition. In some exemplary embodiments, the surfactant is included in at least about 0.5 wt. %, or at least about 0.75 wt. %, or at least about 1.0 wt. %, or at least about 2.0 wt. %, based on the weight of the topical cleansing composition. In one or more exemplary embodiments, the topical cleansing composition comprises up to about 25.0 wt. %, or up to about 18.0 wt. %, or up to about 15.0 wt. %, or up to about 12.0 wt. %, or up to about 9.0 wt. % of one or more surfactants, based on the weight of the topical cleansing composition. In certain exemplary embodiments, the amount of surfactant is from about 2.0 wt. % to about 20.0 wt. %, or from about 2.5 wt. % to about 18.0 wt. %, or from about 3.0 wt. % to about 13.0 wt. %, based on the weight of the topical cleansing composition.

The topical cleansing compositions described herein may be employed in any type of dispenser typically used for gel products, for example pump dispensers. A wide variety of pump dispensers are suitable. Pump dispensers may be affixed to bottles or other free-standing containers. Pump dispensers may be incorporated into wall-mounted dispensers. Pump dispensers may be activated manually by hand or foot pump, or may be automatically activated. Useful dispensers include those available from GOJO Industries under the designations NXT®, TFX™, DPX™, FMX™, ADX™ LTX™, and CXT™ as well as traditional bag-in-box dispensers. Examples of dispensers are described in U.S. Pat. Nos. 5,265,772, 5,944,227, 6,877,642, 7,028,861, 7,611,030, 7,621,426, 8,740,019, 8,991,657, 9,027,790, 9,073,685, 9,101,250, and 9,204,767, all of which are incorporated herein by reference. In one or more embodiments, the dispenser includes an outlet such as a nozzle, through which the topical cleansing composition is dispensed. In some exemplary embodiments, the topical cleansing composition is used in dispensers that employ foaming pumps, which combine ambient air or an inert gas and the composition in a mixing chamber and pass the mixture through a mesh screen.

In one or more embodiments, the topical cleansing composition is integrated into wipe composition. Wipe compositions in accordance with the exemplary embodiments described herein include at least one alcohol, a C₁₋₁₀ alkanediol enhancer, and are applied to a wipe substrate. In some exemplary embodiments, the wipe composition is alcohol-free.

Wipe substrates used in antimicrobial wipes are further described in U.S. Pat. Nos. 5,686,088, 6,410,499, 6,436,892, 6,495,508, 6,844,308, 9,096,821, which are incorporated herein by reference. In one or more embodiments, the wipe may comprise a laminate formed by spunbonding/meltblowing/spunbonding (SMS). Generally, an SMS material contains a meltblown web sandwiched between two exteriors spunbond webs. SMS materials are further described in U.S. Pat. Nos. 4,041,203, 5,169,706, 5,464,688, and 4,766,029, and are commercially available, for example from Kimberly-Clark Corporation under marks such as Spunguard 7 and Evolution 7. The SMS laminate may be treated or untreated.

In some exemplary embodiments, a topical cleansing composition comprising up to about 15.0 wt. % of a polypeptide active ingredient increases the production and/or activity of defensins, such as HBD-1 by a statistically significant amount, as compared to an otherwise identical topical composition that does not include the active ingredient. In some exemplary embodiments, a topical cleansing composition comprising up to about 15.0 wt. % of a polypeptide active ingredient increases the production of defensins, such as HBD-1 by at least 25%, or at least 100%, or at least 500%, or at least 800%, or at least 1000%, as compared to an otherwise identical topical composition that does not include the active ingredient. In some exemplary embodiments, a topical cleansing composition comprising up to about 15.0 wt. % of a polypeptide active ingredient increases the production/activity of defensins, such as HBD-1 by at least 1,400%, or by at least 1,700%, as compared to an otherwise identical topical composition that does not include the active ingredient.

In some exemplary embodiments, a topical cleansing composition comprising up to about 15.0 wt. % of a polypeptide active ingredient increases the production and/or activity of defensins, such as HBD-2 by a statistically significant amount, as compared to an otherwise identical composition that does not include the active ingredient. In some exemplary embodiments, a topical composition comprising up to about 15.0 wt. % of a polypeptide active ingredient increases the production of defensins, such as HBD-2 by at least 25%, or at least 100%, or at least 500%, or at least 800%, or at least 1000%, as compared to an otherwise identical composition that does not include the active ingredient. In some exemplary embodiments, a topical cleansing composition comprising up to about 15.0 wt. % of a polypeptide active ingredient increases the production/activity of defensins, such as HBD-2 by at least 1,100%, or by at least 1,200%, or by at least 2,000%, as compared to an otherwise identical composition that does not include the active ingredient.

In some exemplary embodiments, a topical cleansing composition comprising up to about 15.0 wt. % of a polypeptide active ingredient increases the production and/or activity of defensins, such as HBD-3 by a statistically significant amount, as compared to an otherwise identical composition that does not include the active ingredient. In some exemplary embodiments, a topical composition comprising up to about 15.0 wt. % of an active ingredient increases the production of defensins, such as HBD-3 by at least 25%, or at least 50%, or at least 100%, or at least 500%, or at least 800%, or at least 1000%, as compared to an otherwise identical composition that does not include the active ingredient. In some exemplary embodiments, a topical cleansing composition comprising up to about 15.0 wt. % of a polypeptide active ingredient increases the production/activity of defensins such as HBD-3 by at least 2,000%, or by at least 2,500%, or by at least 4,000%, as compared to an otherwise identical composition that does not include the active ingredient.

In some exemplary embodiments, a topical cleansing composition comprising up to about 15.0 wt. % of an extract active ingredient increases the production and/or activity of defensins, such as HBD-1 by a statistically significant amount, as compared to an otherwise identical composition that does not include the active ingredient. Particularly, a topical cleansing composition comprising up to about 15.0 wt. % of a hydrolysate of linseed proteins increases the production/activity of defensins, such as HBD-1 by at least 10%, or at least 20%, or at least 50%, or at least 75%, or at least 95%, as compared to an otherwise identical composition that does not include the active ingredient.

In some exemplary embodiments, a topical cleansing composition comprising up to about 15.0 wt. % of an extract active ingredient increases the production and or/activity of defensins, such as HBD-2 by a statistically significant amount, as compared to an otherwise identical composition that does not include the active ingredient. Particularly, a topical cleansing composition comprising up to about 15.0 wt. % of a hydrolysate of linseed proteins increases the production/activity of defenins, such as HBD-2 by at least 5%, or at least 10%, or at least 20%, or at least 23%, as compared to an otherwise identical composition that does not include the active ingredient

In some exemplary embodiments, a topical cleansing composition comprising up to about 15.0 wt. % of an extract active ingredient increases the production and/or activity of defensins, such as HBD-3 by a statistically significant amount, as compared to an otherwise identical composition that does not include the active ingredient. Particularly, a topical cleansing composition comprising up to about 15.0 wt. % of a hydrolysate of linseed proteins increases the production/activity of defensins, such as HBD-3 by at least 5%, or at least 10%, or at least 20%, or at least 29%, as compared to an otherwise identical composition that does not include the active ingredient.

In some exemplary embodiments, a topical cleansing composition comprising up to about 15.0 wt. % of an extract active ingredient increases the production and/or activity of cathelicidin-related AMPS, such as LL-37 by a statistically significant amount, as compared to an otherwise identical composition that does not include the active ingredient. Particularly, a topical cleansing composition comprising up to about 15.0 wt. % of a hydrolysate of linseed proteins increases the production/activity of cathelicidin-related AMPS, such as LL-37 by at least 5%, or at least 10%, or at least 20%, or at least 30%, or at least 38%, as compared to an otherwise identical composition that does not include the active ingredient.

In some exemplary embodiments, a topical cleansing composition comprising up to about 15.0 wt. % of an extract active ingredient decreases the production and/or activity of pro-inflammatory factors, such as IL-8 by a statistically significant amount, as compared to an otherwise identical composition that does not include the active ingredient. Particularly, a topical cleansing composition comprising up to about 15.0 wt. % of a hydrolysate of linseed proteins decreases the production/activity of pro-inflammatory factors, such as IL-8 by at least 5%, or at least 10%, or at least 20%, or at least 30%, or at least 33%, as compared to an otherwise identical composition that does not include the active ingredient.

In some exemplary embodiments, a topical cleansing composition comprising up to about 15.0 wt. % of a natural extract active ingredient in a rinse-off increases the production and/or activity of defensins, such as HBD-1 by a statistically significant amount, as compared to an otherwise identical composition that does not include the active ingredient. Particularly, a topical cleansing composition comprising up to about 15.0 wt. % of a hydrolysate of linseed proteins in a rinse-off formulation increases the concentration of HBD-1 by at least 1 pg/mL, or at least 4 pg/mL, or at least 6 pg/mL, or at least 10 pg/mL, or at least 16 pg/mL, as compared to an otherwise identical composition that does not include the active ingredient.

In some exemplary embodiments, a topical cleansing composition comprising up to about 15.0 wt. % of a natural extract active ingredient in a rinse-off formulation increases the production and/or activity of defensins, such as HBD-2 by a statistically significant amount, as compared to an otherwise identical composition that does not include the active ingredient. Particularly, a topical cleansing composition comprising up to about 15.0 wt. % of a hydrolysate of linseed proteins in a rinse off formulation increases the concentration of HBD-2 by at least 1 pg/mL, or at least 10 pg/mL, or at least 25 pg/mL, or at least 40 pg/mL, or at least 60 pg/mL, as compared to an otherwise identical composition that does not include the active ingredient.

In some exemplary embodiments, a topical cleansing composition comprising up to about 15.0 wt. % of a natural extract active ingredient in a rinse-off formulation increases the production and/or activity of defensins, such as HBD-3 by a statistically significant amount, as compared to an otherwise identical composition that does not include the active ingredient. Particularly, a topical cleansing composition comprising up to about 15.0 wt. % of a hydrolysate of linseed proteins in a rinse-off formulation increases the concentration of HBD-3 by at least 1 pg/mL, or at least 50 pg/mL, or at least 100 pg/mL, or at least 150 pg/mL, or at least 185 pg/mL, as compared to an otherwise identical composition that does not include the active ingredient.

### EXAMPLES

The following examples are included for purposes of illustration and are not intended to limit the scope of the methods described herein.

### Example 1

To determine the optimal dose of active ingredient, test dose response studies were run using both Decorinyl® and Pamitoyl Pentapeptided-3. These test dose response studies were commissioned to determine the concentration of HBD-1 at various levels of the active ingredients. Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). NHEK were seeded into 96-well plates at a density of 10000 cells in 200 μl medium per well. After 48 hours, the cells were incubated with varying concentrations of each ingredient solution in a culture medium (KGM) overnight (16 hours) at 37° C., 5% CO₂ and 95% humidity at four replicates for each concentration. Each of these active ingredients was tested at the following weight percents based on the weight of the total culture: 0.02 wt. %, 0.05 wt. %, 0.1 wt. %, 0.2 wt. %, 0.5 wt. %, 1.0 wt. %, 2.0 wt. %. Each of these compositions was compared to a control culture medium.

HBD-1 was detected using HBD-1 ELISA (enzyme-linked immunosorbent assay) developing kits (commercially available from Peprotech). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The results were measured using a colorimeter, absorbance was measured at 450 nanometers (nm) within 30 minutes. Wavelength correction was set to 570 nm. The concentration of each sample was calculated using ELISA standard curve.

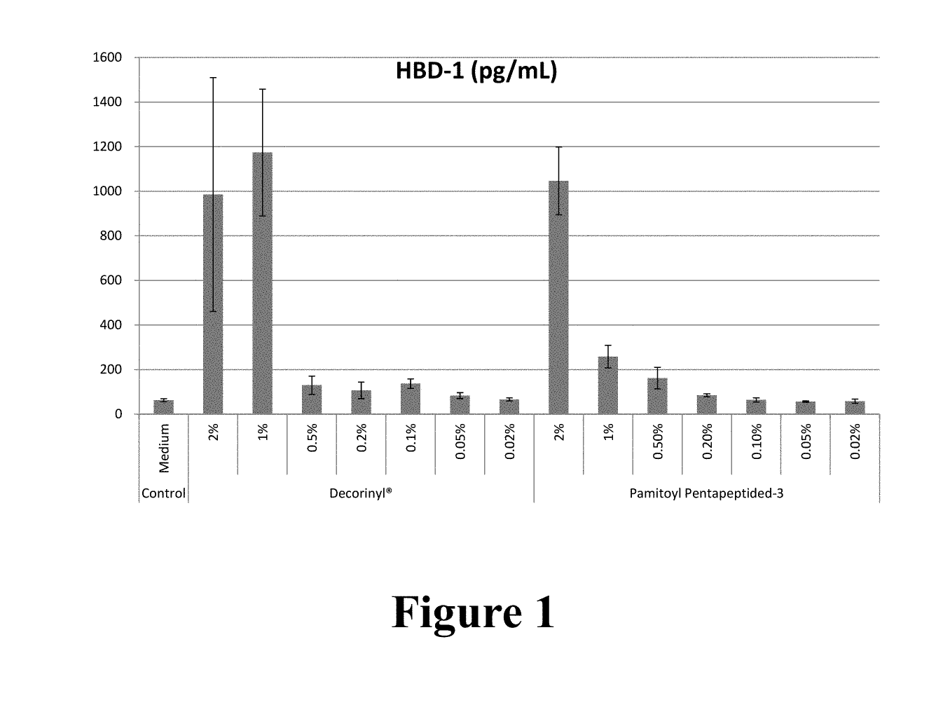

The results are listed below in Table 1 and depicted graphically in FIG. 1. As illustrated below, a 1.0 and 2.0 wt. % concentration of Decorinyl® demonstrated an increase in HBD-1 concentration of 1763% and 1465% were observed for 1.0 wt. % and 2.0 wt. % Decorinyl®, respectively. Increases in HBD-1 concentration of 311% and 1561% were observed for 1.0 wt. % and 2.0 wt. % Pamitoyl Pentapeptided-3, respectively.

TABLE 1

| Active Ingredient | wt. % | HBD-1 (pg/ml) |
|---|---|---|
| Control | Medium | 63 |
| Decorinyl ® | 2% | 986 |
| | 1% | 1174 |
| | 0.5% | 130 |
| | 0.2% | 107 |
| | 0.1% | 138 |
| | 0.05% | 84 |
| | 0.02% | 67 |
| Pamitoyl Pentapeptided-3 | 2% | 1047 |
| | 1% | 259 |
| | 0.50% | 162 |
| | 0.20% | 85 |
| | 0.10% | 64 |
| | 0.05% | 57 |
| | 0.02% | 59 |

### Example 2

To determine the optimal dose of active ingredient, test dose response studies were run using both Decorinyl® and Pamitoyl Pentapeptided-3. These test dose response studies were commissioned to determine the concentration of HBD-2 at various levels of the active ingredients. Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). NHEK were seeded into 96-well plates at a density of 10000 cells in 200 μl medium per well. After 48 hours, the cells were incubated with varying concentrations of each ingredient solution in a culture medium (KGM) overnight (16 hours) at 37° C., 5% CO₂ and 95% humidity at four replicates for each concentration. Each of these active ingredients was tested at the following weight percents based on the weight of the total culture: 0.02 wt. %, 0.05 wt. %, 0.1 wt. %, 0.2 wt. %, 0.5 wt. %, 1.0 wt. %, 2.0 wt. %. Each of these compositions was compared to a control culture medium.

HBD-2 was detected using HBD-2 ELISA developing kits (commercially available from Peprotech). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The results were measured using a colorimeter, absorbance was measured at 450 nanometers (nm) within 30 minutes. Wavelength correction was set to 570 nm. The concentration of each sample was calculated using ELISA standard curve.

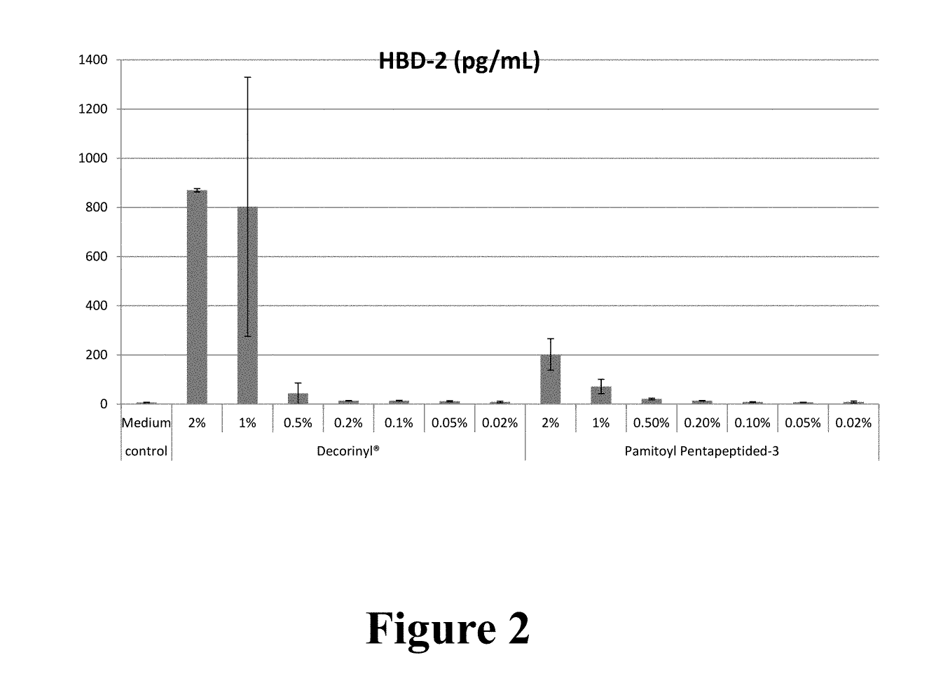

The results are listed below in Table 2 and depicted graphically in FIG. 2. Increases in HBD-2 concentration of 11,371% and 12,329% were observed for 1.0 wt. % and 2.0 wt. % Decorinyl® respectively. An increase in HBD-2 concentration of 2800% was observed for 2.0 wt. % Pamitoyl Pentapeptided-3.

TABLE 2

| Active Ingredient | wt. % | HBD-2 (pg/ml) |
|---|---|---|
| Control | Medium | 7 |
| Decorinyl ® | 2% | 870 |
| | 1% | 803 |
| | 0.5% | 44 |
| | 0.2% | 15 |
| | 0.1% | 15 |
| | 0.05% | 12 |
| | 0.02% | 9 |
| Pamitoyl Pentapeptided-3 | 2% | 203 |
| | 1% | 72 |
| | 0.50% | 21 |
| | 0.20% | 14 |
| | 0.10% | 9 |
| | 0.05% | 8 |
| | 0.02% | 9 |

### Example 3

To determine the optimal dose of active ingredient, test dose response studies were run using both Decorinyl® and Pamitoyl Pentapeptided-3. These test dose response studies were commissioned to determine the concentration of HBD-3 at various levels of the active ingredients. Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). NHEK were seeded into 96-well plates at a density of 10000 cells in 200 μl medium per well. After 48 hours, the cells were incubated with varying concentrations of each ingredient solution in a culture medium (KGM) overnight (16 hours) at 37° C., 5% CO₂ and 95% humidity at four replicates for each concentration. Each of these active ingredients was tested at the following weight percents based on the weight of the total culture: 0.02 wt. %, 0.05 wt. %, 0.1 wt. %, 0.2 wt. %, 0.5 wt. %, 1.0 wt. %, 2.0 wt. %. Each of these compositions was compared to a control culture medium.

HBD-3 was detected using HBD-3 ELISA developing kits (commercially available from Peprotech). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The results were measured using a colorimeter, absorbance was measured at 450 nanometers (nm) within 30 minutes. Wavelength correction was set to 570 nm. The concentration of each sample was calculated using ELISA standard curve.

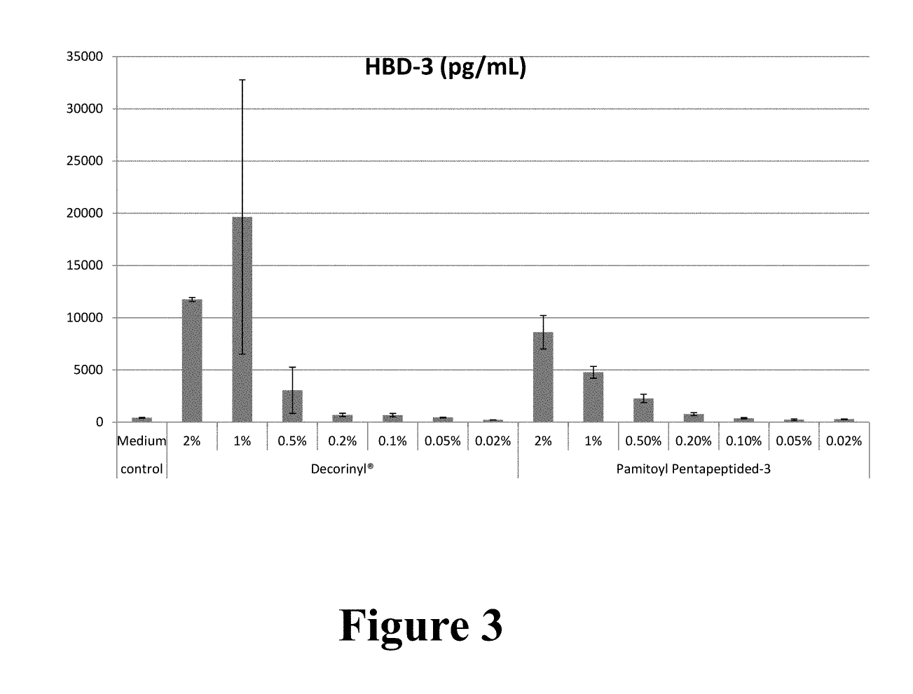

The results are shown below in Table 3 and depicted graphically in FIG. 3. Increases in HBD-3 concentration of 4438% and 2616% were observed for 1.0 wt. % and 2.0 wt. % Decorinyl® respectively. Increases in HBD-3 concentration of 1005% and 1890% were observed for 1.0 wt. % and 2.0 wt. % Pamitoyl Pentapeptided-3, respectively.

TABLE 3

| Active Ingredient | wt. % | HBD-3 (pg/ml) |
|---|---|---|
| Control | Medium | 433 |
| Decorinyl ® | <2% | 11759 |
| | <1% | 19652 |
| | 0.5% | 3058 |
| | 0.2% | 703 |
| | 0.1% | 682 |
| | 0.05% | 456 |
| | 0.02% | 226 |
| Pamitoyl Pentapeptided-3 | <2% | 8617 |
| | <1% | 4783 |
| | 0.50% | 2278 |
| | 0.20% | 775 |
| | 0.10% | 387 |
| | 0.05% | 242 |
| | 0.02% | 288 |

### Example 4

Lipigenine™ was tested for its ability to stimulate an increase in HBD-1 concentration. The HBD-1 standard ABTS (2,2′-Azinobis [3-ethylbenzothiazoline-6-sulfonic acid]-diammonium salt) ELISA development kits were obtained from PeproTech (Cat#900-K202). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The Lipigenine™ culture was compared to the control medium which contained no other ingredients. The results were measured using a colorimeter, absorbance was measured at 450 nanometers (nm) within 30 minutes. Wavelength correction was set to 570 nm. The concentration of each sample was calculated using ELISA standard curve.

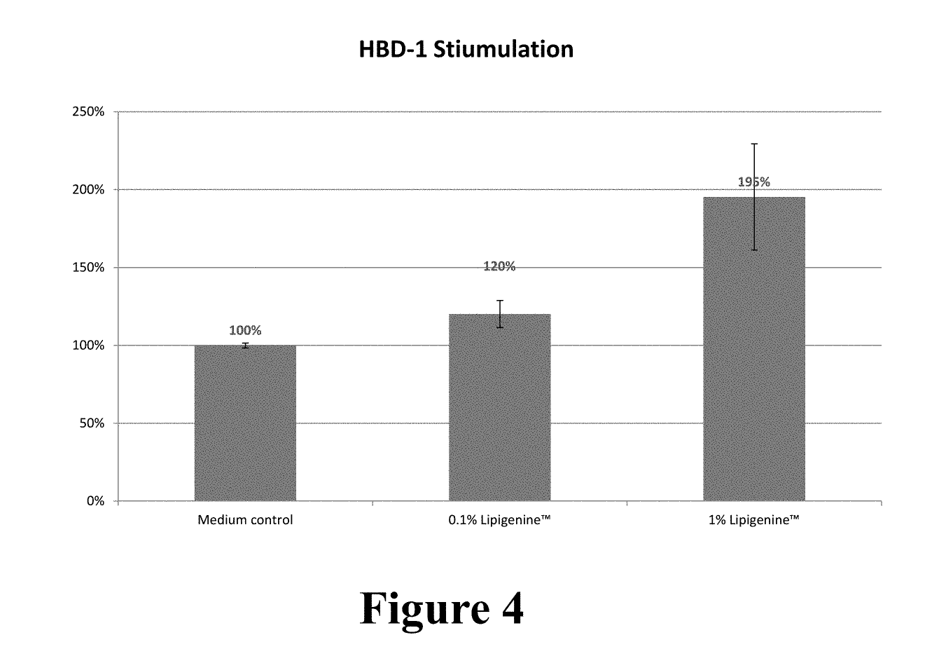

The addition of Lipigenine™ showed high HBD-1 concentration at both 0.1% and 1% Lipigenine™ in solution as compared to the control. An increase in HBD-1 concentration of 20% was observed for 0.1% Lipigenine™ while an increase in HBD-1 concentration of 95% was observed for 1% Lipigenine™. These results are shown in FIG. 4.

### Example 5

Lipigenine™ was tested for its ability to stimulate an increase in HBD-2 concentration. The HBD-2 standard ABTS ELISA development kits were obtained from PeproTech (Cat#900-K172). ELISA was performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The Lipigenine™ culture was compared to the control medium which contained no other ingredients. The results were measured using a colorimeter, absorbance was measured at 450 nanometers (nm) within 30 minutes. Wavelength correction was set to 570 nm. The concentration of each sample was calculated using ELISA standard curve.

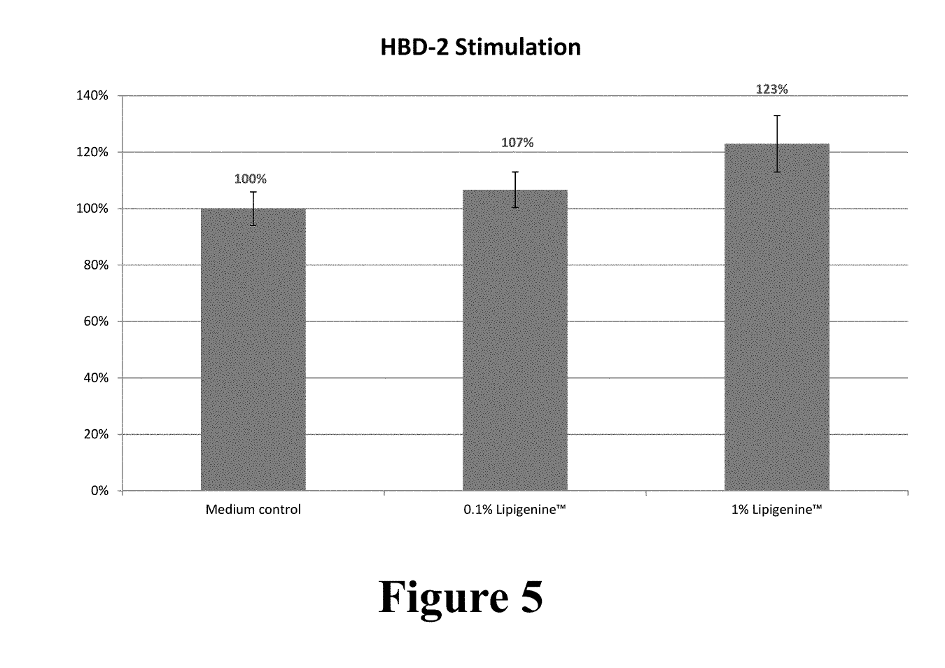

The addition of Lipigenine™ showed increased HBD-2 concentrations at both 0.1% and 1% Lipigenine™ in solution as compared to the control. An increase in HBD-2 concentration of 7% was observed for a 0.1% Lipigenine™ formulation while an increase in HBD-2 expression of 23% was observed for a 1% Lipigenine™ formulation. These results are shown in FIG. 5.

### Example 6

Lipigenine™ was tested for its ability to stimulate an increase in HBD-3 concentration. The HBD-3 standard ABTS ELISA development kit was obtained from PeproTech (Cat#900-K210). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The Lipigenine™ culture was compared to the control medium which contained no other ingredients. The results were measured using a colorimeter, absorbance was measured at 450 nanometers (nm) within 30 minutes. Wavelength correction was set to 570 nm. The concentration of each sample was calculated using ELISA standard curve.

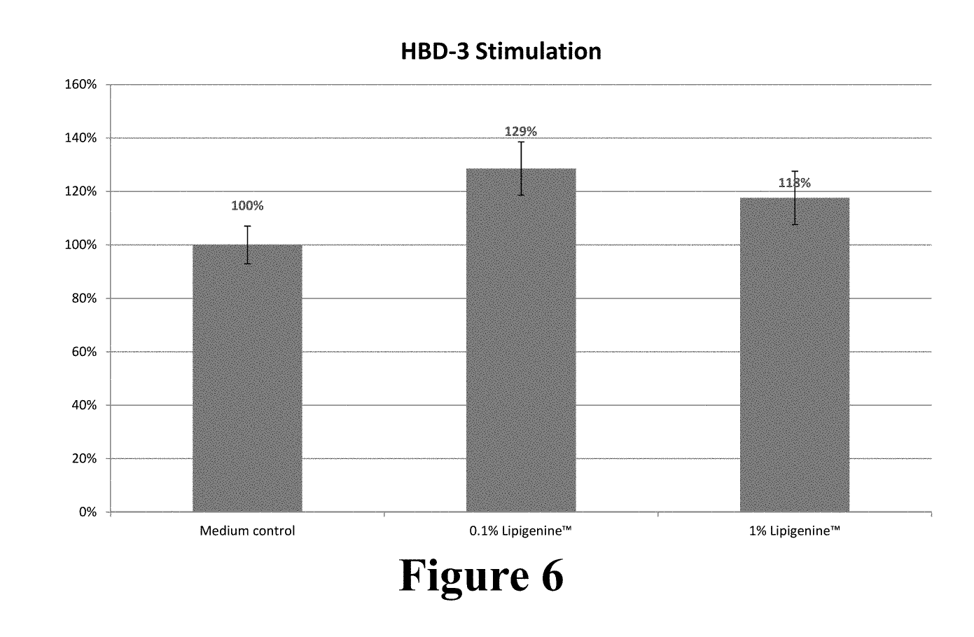

The addition of Lipigenine™ showed increased HBD-3 concentration at both 0.1% and 1% Lipigenine™ in solution as compared to the control. An increase in HBD-3 concentration of 29% was observed for a 0.1% Lipigenine™ formulation while an increase in HBD-3 concentration of 18% was observed for a 1% Lipigenine™ formulation. These results are shown in FIG. 6.

### Example 7

A topical composition with Lipigenine™ was tested for its ability to increase concentration of Cathelicidin (LL37), an amphipathic alpha-helical peptide that plays an important role in defense against local infection and invasion of pathogens at sites of inflammation and wounds. The human LL-37 ELISA kit was obtained from Hycult Biotech (Cat#HK321). ELISA were performed according to the manufactory instructions of each kit by adding 100 μl/well of culture medium after overnight treatment. The results were measured using a colorimeter, absorbance was measured at 450 nanometers (nm) within 30 minutes. Wavelength correction was set to 570 nm.

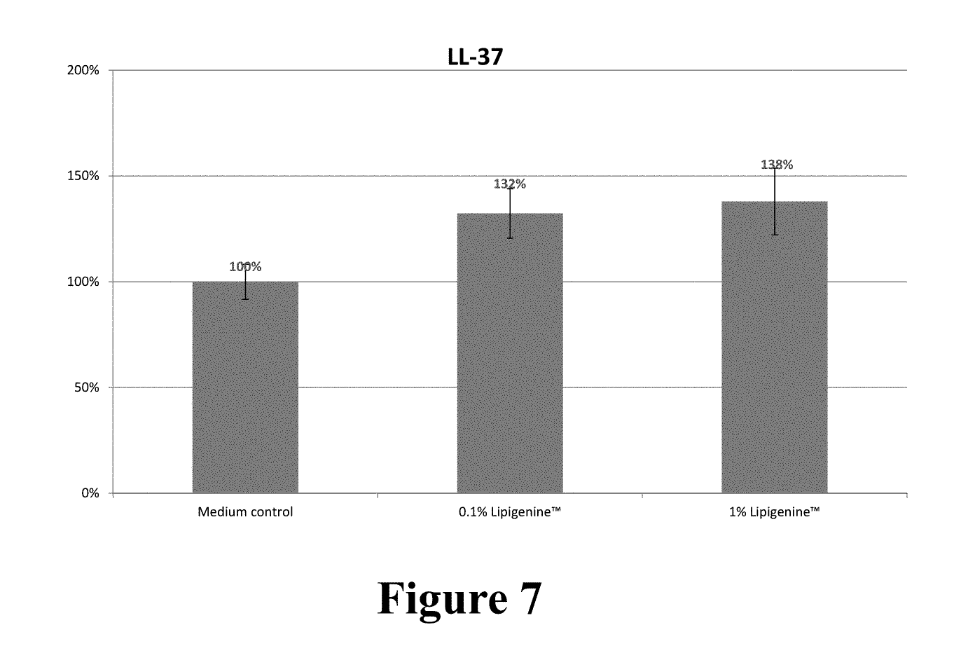

The addition of Lipigenine™ showed increased LL-37 concentration at both 0.1% and 1% Lipigenine™ in solution as compared to the control. An increase in LL-37 concentration of 32% for a 0.1% Lipigenine™ formulation while an increase in LL-37 concentration of 38% was observed for a 1% Lipigenine™ formulation. These results are shown in FIG. 7.

### Example 8

A topical composition with Lipigenine™ was tested for its ability to decrease concentration of Interleukin 8 (IL-8 or CXCL8) which is a chemokine and proinflammatory cytokine produced by macrophages and other cell types such as epithelial cells. It is secreted from keratinocytes in skin in response to inflammatory stimuli. IL-8 is secreted and is an important mediator of the immune reaction in the innate immune system response. IL-8 over-expressed is a biomarker of skin irritation. IL-8 is associated with inflammation and plays a role in colorectal cancer.

For Control A, human dermal keratinocytes were left untreated. No irritation is expected, and therefore Control A provides a baseline (set as 0). For Control B, IL-8 is induced in human dermal keratinocytes by applying a surfactant mixture that is a combination of sodium laureth sulfate and polyquaternium-10 (set as 100%). For all other samples, the human dermal keratinocytes are co-treated with the surfactant mixture and a composition containing indicated concentration of Lipigenine™. Decreased IL-8 expression reflects an ingredient's anti-irritation activity. In order to carry out the test method, an assay kit was employed that was obtained from R&D Systems: Human CXCL8/IL-8 Duoset ELISA Kit (DY208). ELISA was performed after overnight treatment using by applying 100 μl/well of culture medium according to the manufactory instruction of the ELISA kit. The results were measured using a colorimeter, absorbance was measured at 450 nanometers (nm) within 30 minutes. Wavelength correction was set to 570 nm.

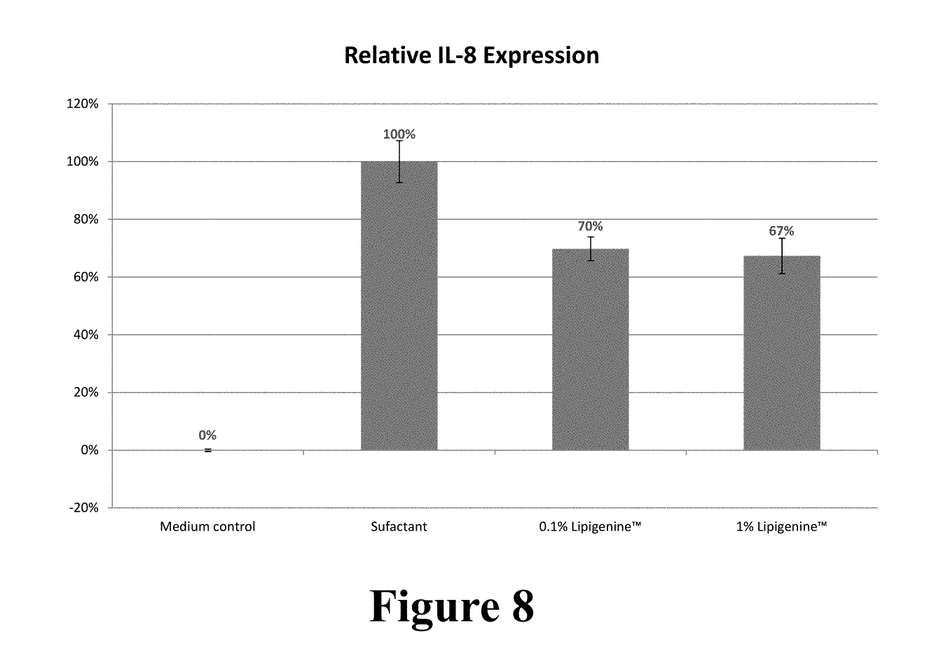

The addition of Lipigenine™ showed reduced IL-8 concentration at both 0.1% and 1% Lipigenine™ in solution as compared to a surfactant. A decrease in IL-8 concentration of 30% was observed for a 0.1% Lipigenine™ formulation while a decrease in IL-8 concentration of 33% was observed for a 1% Lipigenine™ formulation. These results are shown in FIG. 8.

### Example 9

Tape stripping tests were also performed with 5% Lipigenine™ in a soap base formulation (rinse-off) to determine the concentration of AMPs including HBD-1, HBD-2, and HBD-3 on the skin as compared to a soap base without Lipigenine™ (rinse-off). A higher concentration of Lipigenine™ was needed in this example because the formulation was being washed off of the skin instead of being left on. Seven (7) layers of tape strips were applied to the skin at two adjacent sites for both the soap base with Lipigenine™ and the soap base without Lipigenine™. The strips were applied after the two soap bases had been used to clean each skin site. After application, the first layer of tape was discarded as there was too much noise to properly analyze the strip. Thereafter, layers 2-4 were combined (the “Upper Layers”) and layers 5-7 were combined (the “Lower Layers”). These tape striping experiments were run at 0 days (before application), 5 days after application, and 10 days after application to observe increases in AMP concentration over time. Each of the Upper Layers and the Lower Layers were placed in a glass vial and frozen until analysis. Increases in HBD-1 concentration of about 13 pg/mL and about 16 pg/mL were observed for the Upper Layers after 5 days and the Lower Layers after 5 days, respectfully for the soap base with Lipigenine™ as compared to a soap base without Lipigenine™. A statistically significant (95% confidence) increase in HBD-2 concentration of about 63 ng/mL was observed after 5 days in the Lower Layers for the soap base with Lipigenine™ as compared to a soap base without Lipigenine™. A statistically significant (90% confidence) increase in HBD-3 concentration of over 189 pg/mL in HBD-3 was observed after 5 days in the Lower Layers for the soap base with Lipigenine™ as compared to a soap base without Lipigenine™. These results are shown below in Table 4.

TABLE 4

| | | | |
|---|---|---|---|
| | 5% Lipigenine ™ in | (Soap Control) | (Standard |
| | ALSO Soap | ALSO Soap | Control) |
| | Base used as | Base used as | Untreated Skin |
| Layer/Day | a rinse-ff (Code-W) | a rinse-off (Code-R) | Control (Code-U) |

HBD-1 Concentration (pg/mL)
2-4 Upper Layers

| | | | |
|---|---|---|---|
| 0 days | 0 | 0 | 0 |
| 5 days | −7.915 | −5.004 | 0.000 |
| 10 days | −2.209 | 1.696 | 0.000 |

5-7 Lower Layers

| | | | |
|---|---|---|---|
| 0 days | 0 | 0 | 0 |
| 5 days | 9.904 | −6.579 | 0.000 |
| 10 days | 5.223 | −1.794 | 0.000 |

HBD-2 Concentration (pg/mL)
2-4 Upper Layers

| | | | |
|---|---|---|---|
| 0 days | 0 | 0 | 0 |
| 5 days | −26.890 | −17.583 | 0.000 |
| 10 days | −7.192 | 10.595 | 0.000 |

5-7 Lower Layers

| | | | |
|---|---|---|---|
| 0 days | 0 | 0 | 0 |
| 5 days | 35.334 R | −27.588 | 0.000 |
| 10 days | 27.552 | −7.822 | 0.000 |

HBD-3 Concentration (pg/mL)
2-4 Upper Layers

| | | | |
|---|---|---|---|
| 0 days | 0 | 0 | 0 |
| 5 days | 22.321 | −51.342 | 0.000 |
| 10 days | 59.166 | 1.666 | 0.000 |

5-7 Lower Layers

| | | | |
|---|---|---|---|
| 0 days | 0 | 0 | 0 |
| 5 days | 168.683 r | −21.325 | 0.000 |
| 10 days | 141.267 | 22.110 | 0.000 |

Although embodiments of the invention have been described herein, it should be appreciated that many modifications can be made without departing from the spirit and scope of the general inventive concepts. All such modifications are intended to be included within the scope of the invention, which is to be limited only by the following claims.

1. A topical cleansing composition for stimulating the production and/or activity of antimicrobial peptides, the composition comprising;
about 0.005 wt. % to about 15.0 wt. % of an active ingredient; and
at least one primary and at least one secondary surfactant;
wherein the active ingredient comprises one or more of an extract and a polypeptide, wherein said topical cleansing composition increases the production and/or activity of defensins by at least about 7%, relative to an otherwise identical topical composition without said active ingredient.

2. The topical cleansing composition of claim 1, wherein the primary surfactant is sodium laureth sulfate.

3. The topical cleansing composition of claim 1, wherein the secondary surfactant is selected from one or more of cocamidopropyl betaine, disodium cocoamphodiacetate, cocamidopropyl hydroxysultaine, and lauryl glucoside.

4. The topical cleansing composition of claim 1, wherein the extract is one or more of a plant extract, a seed extract, and a fruit extract.

5. The topical cleansing composition of claim 1, wherein the extract is a seed extract.

6. The topical cleansing composition of claim 5, wherein the seed extract is at least one of linseed extract, flaxseed extract, hemp seed extract, grape seed extract, and grapefruit seed extract.

7. The topical cleansing composition of claim 1, wherein the extract is a hydrolysate of proteins.

8. The topical cleansing composition of claim 7, wherein the hydrolysate of proteins is a hydrolysate of proteins extracted from linseed seeds.

9. The topical cleansing composition of claim 8, wherein the hydrolysate of linseed proteins contains from about 0.1 to about 5.0 g/l of peptide compounds and from about 0.1 to about 2.0 g/l of sugar.

10. The topical cleansing composition of claim 9, wherein the peptide compounds have a molecular weight below about 5.0 kDa.

11. The topical cleansing composition of claim 1, wherein the active ingredient is a polypeptide.

12. The topical cleansing composition of claim 11, wherein the polypeptide is at least one of an oligopeptide and a hexapeptide.

13. The topical cleansing composition of claim 1, wherein the topical cleansing composition comprises from about 0.05 to about 5.0 wt. % active ingredient, based on the weight of the topical cleansing composition.

14. (canceled)

15. The topical cleansing composition of claim 1, wherein the topical cleansing composition further comprises one or more skin conditioning agents.

16. The topical cleansing composition of claim 15, wherein the one or more skin conditioning agents comprises one or more humectants, comprising propylene glycol, hexylene glycol, 1,4-dihydroxyhexane, 1,2,6-hexanetriol, sorbitol, butylene glycol, caprylyl glycol, propanediols, such as methyl propane diol, dipropylene glycol, triethylene glycol, glycerin (glycerol), polyethylene glycols, ethoxydiglycol, polyethylene sorbitol, glyceryl caprylate/caprate, and combinations thereof.

17. (canceled)

18. The topical cleansing composition of claim 16 or 17, wherein the humectant is present in an amount up to about 20.0 wt. %, based on the weight of the topical cleansing composition.

19. The topical cleansing composition of claim 1, wherein the topical composition further comprises one or more moisturizing esters, comprising cetyl myristate, cetyl myristoleate, and other cetyl esters, diisopropyl sebacate, isopropyl myristate, and combinations thereof.

20. (canceled)

21. The topical cleansing composition of claim 1, wherein the topical cleansing composition increases the production and/or activity of defensins by at least about 18%, relative to an otherwise identical topical composition without the active ingredient.

22. (canceled)

23. The topical cleansing composition of claim 1, wherein the topical cleansing composition increases the production and/or activity of cathelicidin-related antimicrobial peptides by at least about 32%, relative to an otherwise identical topical composition without the active ingredient.

24. The topical cleansing composition of claim 1, wherein the topical cleansing composition decreases the production and/or activity of chemokines by at least about 30%, relative to an otherwise identical topical composition without the active ingredient.

25. The topical cleansing composition of claim 1, wherein the topical cleansing composition increases the production and/or activity of defensins by at least about 4 pg/mL, relative to an otherwise identical topical composition without the active ingredient.

26. The topical cleansing composition of claim 1, wherein the topical cleansing composition increases the production and/or activity of defensins by at least about 25 pg/mL, relative to an otherwise identical topical composition without the active ingredient.

27. The topical cleansing composition of claim 1, wherein the topical cleansing composition further comprises at least one carrier.

28. (canceled)

29. A method of skin treatment to increase the production and/or activity of at least one antimicrobial peptide on the skin, the method comprising:
applying a topical cleansing composition to a skin surface, wherein the topical composition comprises:
about 0.005 wt. % to about 15.0 wt. % of an active ingredient; and
at least one primary and at least one secondary surfactant;
wherein the active ingredient comprises one or more of an extract and a polypeptide, wherein said topical cleansing composition increases the production and/or activity of defensins by at least about 7%, relative to an otherwise identical topical cleansing composition without said active ingredient.
